(19) United States
(12) Patent Application Publication
(10) Pub. No.: US 2014/0073889 A1
(43) Pub. Date: Mar. 13, 2014

(54) SYSTEMS AND METHODS FOR DETERMINING FLUID RESPONSIVENESS

(71) Applicants: Mark Su, Boulder, CO (US); Bo Chen, Louisville, CO (US)

(72) Inventors: Mark Su, Boulder, CO (US); Bo Chen, Louisville, CO (US)

(73) Assignee: Nellcor Puritan Bennett LLC, Boulder, CO (US)

(21) Appl. No.: 13/611,153

(22) Filed: Sep. 12, 2012

(51) Int. Cl. A61B 5/0205; A61B 5/1455

(52) U.S. Cl. 600/324; 600/484

(57) ABSTRACT

A system is provided including a ventilator detection module, a circulatory detection module, and an analysis module. The ventilator detection module is configured to detect ventilator information representative of a ventilation activity. The circulatory detection module is configured to detect circulatory information representative of the circulation of the patient. The analysis module is configured to obtain a ventilator waveform based at least in part on the ventilator information, obtain a circulatory waveform based at least in part on the circulatory information, combine the ventilator waveform and the circulatory waveform to provide a mixed waveform, and isolate a portion of the mixed waveform to identify a ventilator responsiveness waveform representative of an effect of the ventilator.

### FIELD

Embodiments of the present disclosure generally relate to physiological signal processing, and more particularly, to processing signals to determine the fluid responsiveness of a patient.

### BACKGROUND

A physician or nurse may use an index of fluid responsiveness to help determine whether the blood flow of a ventilated patient will benefit from additional fluid administration. Such dynamic indices may be based on a ventilator-induced variation of an arterial-line pressure waveform or a photoplethysmographic (“PPG”) waveform. Waveform variation may be caused by the following: 1) a ventilator cycle induces a cyclic increase in intrathoracic pressure, which causes 2) a cyclic reduction in venous return, which in turn causes 3) a cyclic reduction in preload, which causes 4) a cyclic reduction in cardiac output, which is manifested as 5) a cyclic variation in the arterial line pressure or PPG waveform. A large waveform variation indicates that cardiac output can likely be increased with fluid administration.

However, dynamic indices based on waveform variation are fluid-response predictive only at relative extremes of large waveform variation induced by high-tidal-volume ventilation. The use of lung-protective ventilation strategies for patients with acute lung injury (ALI) or acute respiratory distress syndrome (ARDS) means that many of the most critical patients do not have a large enough ventilation-induced waveform variation to use as a fluid-responsiveness measure with certain known techniques. Further still, the interpretation of dynamic indices or measurements used to arrive at such interpretations may be confounded by a number of factors. For example, artifacts introduced into the signal by sources other than the ventilator-induced changes in intrathoracic pressure may confound the analysis. As another example, differences in ventilator mode, circuit impedance, pressure and flow settings can all affect the size of the ventilator-induced waveform variability. A need exists for improved determination of fluid responsiveness.

### SUMMARY

Certain embodiments provide a system that may include a ventilator detection module, a circulatory detection module, and a fluid responsiveness analysis module. The ventilator detection module may be configured to be operably connected to a ventilator, and to detect ventilator information representative of a ventilation activity performed by the ventilator on a ventilated patient. The ventilator information corresponds to one or more of a pressure of the ventilator, a flow of the ventilator, or a volume of the ventilator. The circulatory detection module may be configured to detect circulatory information representative of the circulation of the ventilated patient. The fluid responsiveness analysis module may be configured to obtain a ventilator waveform based at least in part on the ventilator information from the ventilator detection module, obtain a circulatory waveform based at least in part on the circulatory information from the circulatory detection module, combine the ventilator waveform and the circulatory waveform to provide a mixed waveform, and isolate a portion of the mixed waveform to identify a ventilator responsiveness waveform representative of an effect of the ventilator on the mixed waveform, and determine a fluid responsiveness parameter representative of fluid responsiveness of the ventilated patient using the ventilator responsiveness waveform.

The analysis module may be further configured to determine a fluid responsiveness parameter representative of fluid responsiveness of the ventilated patient using the ventilator responsiveness waveform.

The analysis module may be further configured to combine the circulatory waveform and the ventilator waveform by multiplication, and to isolate the portion of the mixed waveform by applying a low-pass filter.

In some embodiments, the circulatory detection module may include a pulse oximetry sensor configured to provide photoplethysmographic information representative of a photopleythsmographic waveform of the ventilated patient. In some embodiments, the circulatory detection module may include an arterial line catheter and a pressure transducer. The pressure transducer may be configured to be associated with the arterial line catheter and to provide blood pressure information representative of a blood pressure waveform of the ventilated patient.

The ventilator information may include information corresponding to a variation in positive end expiratory pressure (PEEP) of the ventilator.

Certain embodiments provide a method for determining fluid responsiveness. The method includes obtaining a ventilator waveform representative of a ventilation activity performed by a ventilator on a ventilated patient. The method also includes obtaining a circulatory waveform representative of the circulation of the ventilated patient. The circulatory waveform is based on information provided by a circulatory detection module. The method further includes combining, at a processing module, the ventilator waveform and the circulatory waveform to provide a mixed waveform. Further, the method includes isolating, at the processing module, a portion of the mixed waveform to provide a ventilator responsiveness waveform representative of an effect of the ventilator on the mixed waveform.

Certain embodiments provide a tangible and non-transitory computer readable medium including one or more computer software modules. The one or more computer software modules are configured to direct a processor to obtain a ventilator waveform representative of a ventilation activity perform by a ventilator on a ventilated patient. Also, the computer software module(s) may be configured to direct a processor to obtain a circulatory waveform representative of the circulation of the ventilated patient. The circulator waveform may be based on information provided by a circulatory detection module. Further, the computer software module(s) may be configured to direct a processor to combine the ventilator waveform and the circulatory waveform to provide a mixed waveform, and to isolate a portion of the mixed waveform to provide a ventilator responsiveness waveform representative of an effect of the ventilator on the mixed waveform.

Embodiments provide systems and methods configured to isolate ventilation variability (variability caused by ventilation in a waveform) from other variability (variability caused by one or more other sources of potential variability), thereby allowing for a more controlled study and determination of fluid responsiveness. For example, embodiments provide systems and methods that are configured to more accurately determine a fluid responsiveness index or indices. Also, embodiments provide improved predictive value of fluid responsiveness determinations. Further, embodiments provide systems and methods that are configured to allow a determination of fluid responsiveness at relatively low tidal volume ventilation. Also, embodiments provide systems and methods configured to determine fluid responsiveness for smaller variations of waveforms.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

FIG. 1 illustrates a schematic diagram of a system for determining fluid responsiveness in accordance with an embodiment.

FIG. 2 illustrates an example of a composite ventilator waveform in accordance with an embodiment.

FIGS. 3a and 3b illustrate a mixed waveform in accordance with an embodiment.

FIG. 4 illustrates an isometric view of a photoplethysmogram (PPG) system in accordance with an embodiment.

FIG. 5 illustrates a simplified block diagram of a PPG system in accordance with an embodiment.

FIG. 6 illustrates a flowchart of a method for determining fluid responsiveness in accordance with an embodiment.

FIG. 7 illustrates a PPG signal in accordance with an embodiment.

FIG. 8 illustrates a depiction of signal variability in accordance with various embodiments.

FIG. 9 illustrates a flowchart of a method for determining fluid responsiveness in accordance with an embodiment.

### DETAILED DESCRIPTION

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Embodiments of the present disclosure provide systems and methods configured to isolate ventilation variability (variability caused by ventilation in a waveform) from other variability (variability caused by one or more other sources of potential variability), thereby allowing for a more controlled study and determination of fluid responsiveness. For example, embodiments provide systems and methods that are configured to more accurately determine a fluid responsiveness index or indices. Further, embodiments provide systems and methods that are configured to allow a determination of fluid responsiveness at relatively low tidal volume ventilation. Also, embodiments provide systems and methods configured to determine fluid responsiveness for smaller variations of waveforms.

Fluid responsiveness relates to the volume of fluid, such as blood, in the arteries, veins, and vasculature of an individual. In general, fluid responsiveness may include a measurement of the response of stroke volume, the volume of blood passing out of the heart with each heartbeat, to venous return, the volume of blood entering the heart with each heartbeat, caused by the clinical administration of fluid into the vasculature, such as through an intravenous injection. With each heartbeat, a certain amount of blood is pumped out of the heart. The more blood that fills the heart, the more blood the heart can pump out with each heartbeat. If blood volume is too low, the heart may not fully fill with blood. Therefore, the heart may not pump out as much blood with each heartbeat. Consequently, low blood volume may lead to low blood pressure, and organs and tissues may not receive enough blood to optimally and/or properly function. Monitoring fluid responsiveness allows a physician to determine whether additional fluid should be provided to a patient, such as through an intravenous fluid injection. In short, fluid responsiveness represents a prediction of whether or not additional intravenous fluid may improve blood flow within a patient. Fluid responsiveness may be viewed as a response of a heart in relation to overall fluid within a patient.

Fluid responsiveness may be monitored in, for example, critically-ill patients because fluid administration plays an important role in optimizing stroke volume, cardiac output, and oxygen delivery to organs and tissues. However, clinicians need to balance central blood volume depletion and volume overloading. Critically-ill patients are generally at greater risk for volume depletion and severe hypotension is a common life-threatening condition in critically-ill patients. Conversely, administering too much fluid may induce life-threatening adverse effects, such as volume overload, systemic and pulmonary edema, and increased tissue hypoxia. Therefore, obtaining reliable information and parameters that aid clinicians in fluid management decisions may help improve patient outcomes.

An index (e.g. a unitless parameter or percentage) of fluid responsiveness, or index of dynamic preload responsiveness, may be used, to help determine whether the blood flow of a ventilated patient will benefit from additional fluid administration. Such an index may be used to describe a variability corresponding to fluid responsiveness. For example, stroke volume variation (SVV; which may be defined as (SVₘₐₓ−SVₘᵢₙ)/SVₘₑₐₙ over a respiratory cycle) and pulse pressure variation (PPV; which may be defined as automated pulse pressure variations expressed as a percentage) are indices that may currently be obtained using arterial-line pressure waveforms, and the pleth variability index (PVI; which may be defined as (PIₘₐₓ−PIₘᵢₙ)/PIₘₐₓ, where PI=(AC_{IR}/DC_{IR})×100) is an index that may obtained using a PPG. For example, when such an index exceeds a predetermined threshold (e.g. 10%, 15%, or other threshold), additional fluid administration may be indicated. However, use of such indices obtained using current methods may generally be supported at higher tidal volumes. For example, SVV obtained by current methods may generally be supported for patients who are 100% mechanically ventilated with tidal volumes of more than 8 cc/kg and fixed respiratory rates. As discussed herein, embodiments of the present disclosure are configured to isolate, on the one hand, the variability in a measured physiological (e.g. circulatory) parameter due to mechanical or induced ventilation from, on the other hand, variability due to other sources. The isolation provides improved accuracy, sensitivity, and/or reliability of determined fluid responsiveness. Additionally, the isolation allows use of lower tidal volumes in ventilated patients when determining fluid responsiveness.

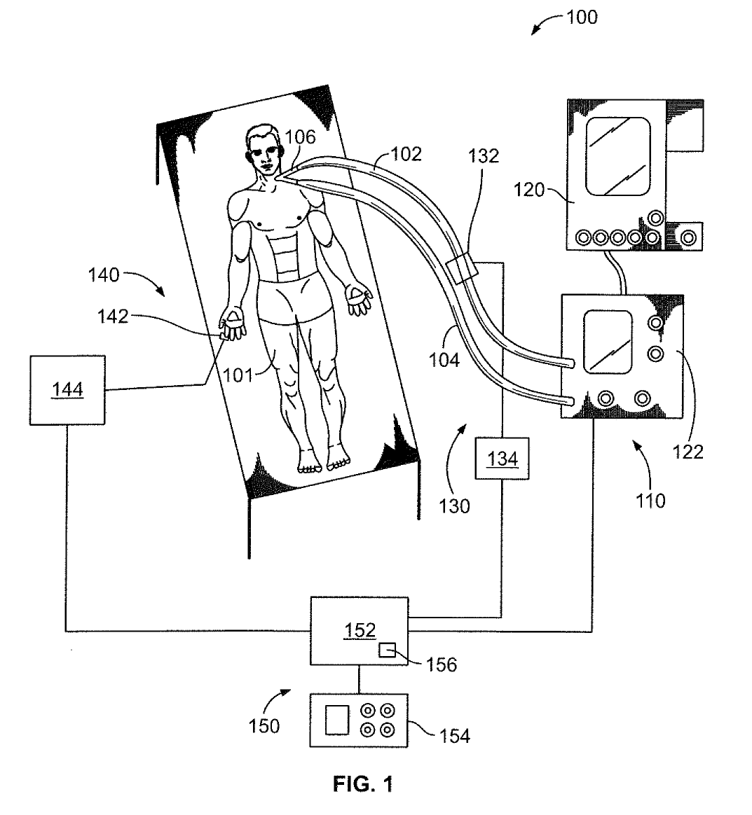

FIG. 1 illustrates a schematic diagram of a system 100 for determining fluid responsiveness in accordance with various embodiments. The system 100, for example, may be used in conjunction with embodiments or aspects of methods described elsewhere herein. The system 100 includes a ventilator system 110, a ventilator detection module 130, a physiological detection module 140, and a fluid responsiveness analysis module 150. In the illustrated embodiment, the fluid responsiveness analysis module 150 is configured to determine fluid responsiveness (e.g. a parameter such as an index representative of the fluid responsiveness of the patient 101) using information provided by the ventilator system 110, the ventilator detection module 130, and the physiological detection module 140.

The various systems, modules, and units disclosed herein may include a controller, such as a computer processor or other logic-based device that performs operations based on one or more sets of instructions (e.g., software). The instructions on which the controller operates may be stored on a tangible and non-transitory (e.g., not a transient signal) computer readable storage medium, such as a memory. The memory may include one or more computer hard drives, flash drives, RAM, ROM, EEPROM, and the like. Alternatively, one or more of the sets of instructions that direct operations of the controller may be hard-wired into the logic of the controller, such as by being hard-wired logic formed in the hardware of the controller.

In the embodiment illustrated in FIG. 1, a patient 101 is shown receiving ventilation therapy from the ventilator system 110. The ventilator system 110 includes a graphic user interface 120 operably connected to and controlling a breath delivery unit, or respirator 122. The patient 101 is connected to the respirator 122 by a patient circuit including an inspiratory line 102, an expiratory line 104, and a patient connection tube 106, all connected by a patient connector (not shown) of a type known to one of ordinary skill in the art. The respirator 122 includes a processor or controller configured to control the real-time operation of the respirator 122. For example, the processor or controller may provide a specific pressure, volume, flow, and/or PEEP to the patient 101 responsive to values input by a practitioner using the graphic user interface 120. In the illustrated embodiment, the respirator 122 also includes internal detectors that sense one or more outputs or characteristics of the ventilator system 110, such as a pressure or flow of air provided to the patient 101, and provides information representative of the sensed characteristics to the fluid responsiveness analysis module 150. For example, the information may include collected data used by the fluid responsiveness analysis module 150 to construct one or more waveforms describing the sensed characteristics or outputs. In other embodiments, the information may include a waveform, either in a raw or processed form (e.g. filtered or normalized) provided to the fluid responsiveness analysis module 150.

The ventilator detection module 130 is configured to sense one or more outputs or characteristics of the ventilator system 110, such as a pressure or flow of air provided to the patient 101, and to provide information representative of the sensed characteristics to the fluid responsiveness analysis module 150. For example, in the illustrated embodiment, the ventilator detection module 130 includes a ventilator detector 132 and a ventilator detector processing unit 134. The ventilator detector 132 is configured to detect a property or output of the ventilator system 110, and to provide information representative of the detected property or output to the ventilator detector processing unit 134. For example, the ventilator detector 132 may include a valve or flow meter operably connected to the inspiration line 102.

The ventilator detector processing unit 134 then constructs and processes (e.g. filters or normalizes) a waveform using information provided by the ventilator detector 132, and in turn provides the waveform to the fluid responsiveness analysis module 150. Further still, the ventilator detector processing unit 134 may include a display and/or user interface allowing adjustment or selection of modes of processing of a ventilation waveform constructed using information provided by the ventilator detector 132. In other embodiments, the ventilator detector 132 may provide the information directly to the fluid responsiveness analysis module 150, with some or all of the functionality of the ventilator detector processing unit 134 incorporated into the fluid responsiveness analysis module 150. In some embodiments, information is provided to the fluid responsiveness analysis module 150 by both the ventilator system 110 and the ventilator detection module 130. In other embodiments, information may be provided to the fluid responsiveness analysis module 150 from only one of the ventilator system 110 or the ventilator detection module 130. For example, in some embodiments, a ventilator detection module 130 configured to be positioned externally of the ventilator system 110 may not be provided.

The physiological detection module 140 is configured to sense one or more physiological characteristics of the patient 101, and to provide information representative of the sensed characteristics to the fluid responsiveness analysis module 150. For example, the physiological detection module 140 in some embodiments is configured to detect a circulatory characteristic of the patient 101, such as a PPG or, as another example, an arterial line pressure. In the illustrated embodiment, the physiological detection module 140 includes a physiological detector 142 and a physiological detector processing unit 144. The physiological detector 142 is configured to detect a physiological property or characteristic of the patient 101, and to provide information representative of the detected property or characteristic to the physiological detector processing unit 144. For example, in the illustrated embodiment, the physiological detector 142 includes a pulse oximeter configured for placement proximal to a finger of the patient 101 as depicted in the illustrated embodiment. The physiological detector processing unit 144 then constructs and processes (e.g. filters or normalizes) a waveform using information provided by the physiological detector 142, and in turn provides the waveform to the fluid responsiveness analysis module 150. Further still, the physiological detector processing unit 144 may include a display and/or user interface allowing adjustment or selection of modes of processing of a physiological waveform constructed using information provided by the physiological detector 142. In other embodiments, the physiological detector 142 may provide the information directly to the fluid responsiveness analysis module 150, with some or all of the functionality of the physiological detector processing unit 144 incorporated into the fluid responsiveness analysis module 150.

The fluid responsiveness analysis module 150 is configured to receive information from the ventilator system 110 and/or ventilator detection module 130 as well as the physiological detection module 140, and to determine a measure or indication of fluid responsiveness using the provided information. The information may be provided in the form of one or more waveforms and/or one or more datasets that may be used to construct a waveform. For example, the fluid responsiveness analysis module 150 may receive ventilator information from the ventilator system 110 and construct a ventilator waveform using the ventilator information.

In some embodiments, the ventilator waveform is a composite waveform. For example, the ventilator waveform may be formed or constructed from a composite of different types of measured or detected ventilator information or waveforms. Generally speaking, a function C(t) describing the ventilator waveform is selected to define a signal (either composite or otherwise) that is best correlated to fluid responsiveness. Such a function C(t) may for example, be determined experimentally through clinical studies. The function C(t) may vary across different patient types or populations, and may also vary based on the type of equipment (e.g. ventilator or sensors) or combination of equipment.

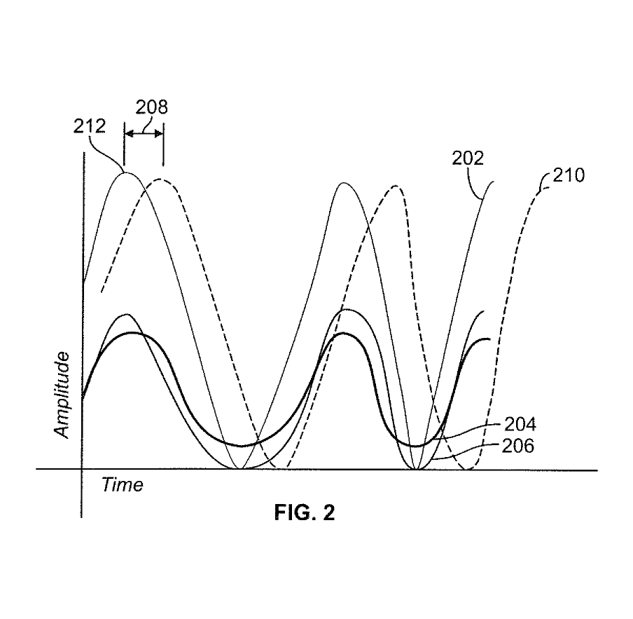

As discussed above, in embodiments, the function C(t) may be a function of a single measured or determined parameter, such as volume or flow, while in other embodiments, the function C(t) may be a composite of measured, determined, or derived waveforms or parameters. FIG. 2 illustrates an example of a composite ventilator waveform 202 in accordance with an embodiment. In the illustrated embodiment, the function C(t) may correspond to ventilator work, and may be described as C(t)=P(t+d)*V(t+d), where C is the ventilator waveform at a given time t, P is the ventilator pressure, V is the ventilator volume, and d is a time delay selected to synchronize the ventilator waveform with another waveform. The composite ventilator waveform 202 (e.g. ventilator work) is formed by multiplying the ventilator pressure waveform 204 by the ventilator volume waveform 206. The particular shapes of the waveforms in FIG. 2 are intended for clarity of illustration and may vary in practice. A time delay 208 may be added to form a synchronized composite ventilator waveform 210 (shown as a dashed line for clarity in FIG. 2) that is shifted by the delay d from the composite ventilator waveform 202. For example, a feature of the composite waveform 202, such as peak 212 may be identified to correlate to a particular event in a physiological cycle. A corresponding portion of an additional waveform (e.g. a PPG waveform as discussed below) may be identified as correlating to the same particular event. The delay 208 may then be computed and applied to the composite waveform 202 to form the synchronized composite ventilator waveform 210 with the additional waveform.

Other composite waveforms using additional or alternative ventilator information may be used in other embodiments. Such composite waveforms may be formed by multiplying different types of ventilator characteristic waveforms, adding different types of ventilator characteristic waveforms, or otherwise. For example, a composite waveform may be obtained by combining one or more measured or determined waveforms, and/or one or more derivatives and/or integrals of one or more measured or determined waveforms. Further, coefficients may be employed to provide different weightings of constituent waveforms used to provide the composite waveform. Specific combinations and coefficients for such composite waveforms may be determined experimentally, for example in clinical studies. For example, different types of ventilator information (e.g. pressure, flow, PEEP) may be collected during a clinical study during which fluid responsiveness is determined by known methods. Then, the different types of ventilator information may be combined in a plurality of combinations, and the results provided by the particular combinations analyzed to determine which combination or combinations of ventilator information are most effective in describing fluid responsiveness.

Further, in some embodiments, the function C(t) may correspond to information collected substantially continuously throughout a physiological cycle, such as a respiratory cycle. As another example, the function C(t) may correspond to information collected at periods or intervals during a physiological cycle. For instance, a function C(t) in accordance with some embodiments may correspond to a variation of PEEP over time

The fluid responsiveness analysis module 150 may also receive physiological information (e.g. PPG information) from the physiological detection module 140 and construct a physiological waveform using the ventilator information. In other embodiments, the fluid responsiveness analysis module 150 may receive one or more waveforms constructed by one or more of the respective detection modules. Further still, the fluid responsiveness analysis module 150, in some embodiments, is configured to process received information and/or waveforms, for example by filtering to remove noise or other artifacts, or, as another example, to synchronize two waveforms to each other.

The fluid responsiveness analysis module 150 is further configured to isolate information representing variability due to ventilation from information representing variability due to other sources. For example, in some embodiments, the fluid responsiveness analysis module 150 is configured to synchronize the ventilator waveform and the physiological waveform, multiply the two waveforms to provide a mixed waveform, and then apply a low pass filter to the mixed waveform to provide a ventilator responsiveness waveform.

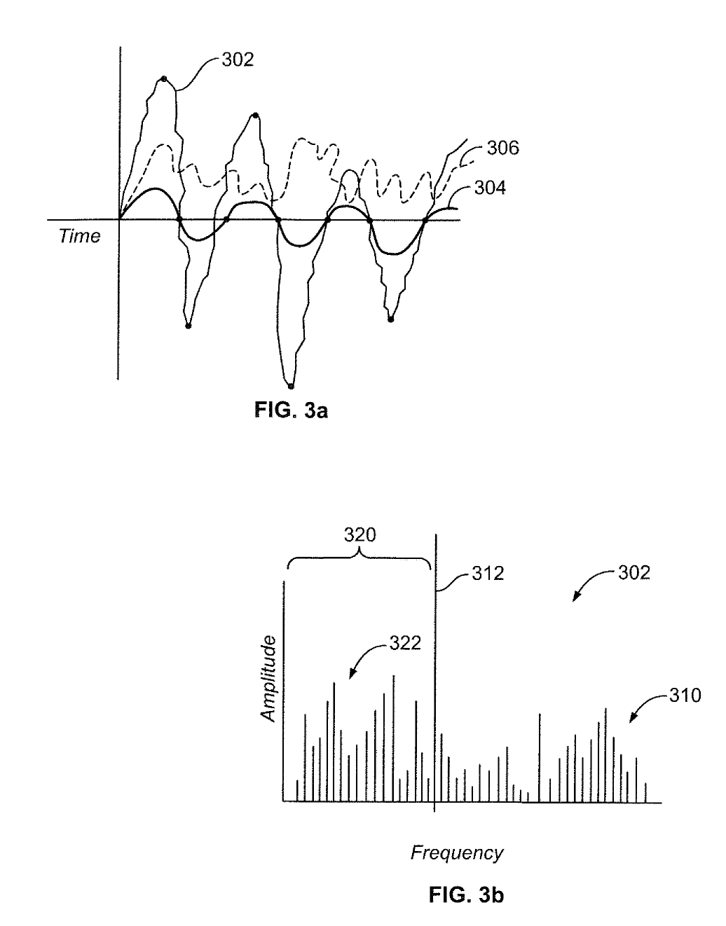

FIGS. 3a and 3b illustrate a mixed waveform 302 in accordance with an embodiment. Two waveforms may be combined to form the mixed waveform 302. For example, in the illustrated embodiment, a composite ventilator waveform 304 and a physiological waveform 306 (shown as a dashed line for clarity) are multiplied to form the mixed waveform 302. For example, the physiological waveform may be a PPG waveform. (See, e.g., discussion below.) The particular shapes of the waveforms in FIGS. 3a and 3b are intended for clarity of illustration and may vary in practice. In FIG. 3b, the mixed waveform 302 is depicted as a spectrum 310 in a frequency domain. A cut-off frequency 312 is depicted. A low-pass filter having a cut-off frequency of 312 may be applied to the mixed waveform 302 to produce a ventilator responsiveness waveform 320 (represented as a spectrum 322 in the frequency domain in FIG. 3b). Referring to FIGS. 1, 3a, and 3b, in the illustrated embodiment, the fluid responsiveness analysis module 150 includes a lock-in detection module 156 configured to multiply the composite ventilator waveform 304 and the physiological waveform 306 to form the mixed waveform 302, and apply a low-pass filter to the mixed waveform 302. For example, the lock-in detection module 156 may comprise a lock-in amplifier.

The variability of the ventilator responsiveness waveform 320 provides an indication of the effect of ventilation partially or entirely separated from other sources of potential variation in the mixed waveform 302 or the physiological waveform 306. The ventilator responsiveness waveform 320 may then be analyzed by the fluid responsiveness analysis module 150, or additionally or alternatively by a practitioner, to determine fluid responsiveness, for example a fluid responsiveness variability index. For example, the variability of the ventilator responsiveness waveform 320 may be analyzed to provide an index that may be correlated by clinical studies to a threshold for determining whether additional fluid administration is appropriate.

In the illustrated embodiment, the fluid responsiveness analysis module 150 is depicted as a stand-alone unit including a processing module 152 and a display module 154. The processing module 152, for example, may be configured to receive a ventilator waveform 304 and a physiological waveform 306, multiply the two waveforms and apply a bandpass filter to obtain a fluid responsiveness waveform 320, and determine a fluid responsiveness parameter using the fluid responsiveness waveform 320. The processing module 152 may, in some embodiments, be further configured to determine a fluid administration recommendation using the fluid responsiveness parameter. The display module 154, for example, may include a graphic user interface that displays a computed measure of ventilator responsiveness variability, such as an index, and/or displays a recommendation regarding whether additional fluid administration is appropriate. The graphic user interface of the display module 154 may also be configured to allow a practitioner to adjust settings of the fluid responsiveness analysis module 150. For example, in some embodiments, the fluid responsiveness analysis module 150 includes a plurality of predetermined composite waveform formulations, and a practitioner may select an appropriate technique for constructing a composite ventilator waveform from among the plurality, for example, based on one or more of patient characteristics (e.g. age, weight) or equipment characteristics (e.g. sensor type, ventilator setting). In still other embodiments, the fluid responsiveness analysis module 150 may be incorporated into a monitor or processing unit that also provides additional functionality. For example, in some embodiments, the fluid responsiveness analysis module 150 may be incorporated into a ventilator system, such as the ventilator system 110 including the respirator 122 and graphic user interface 120.

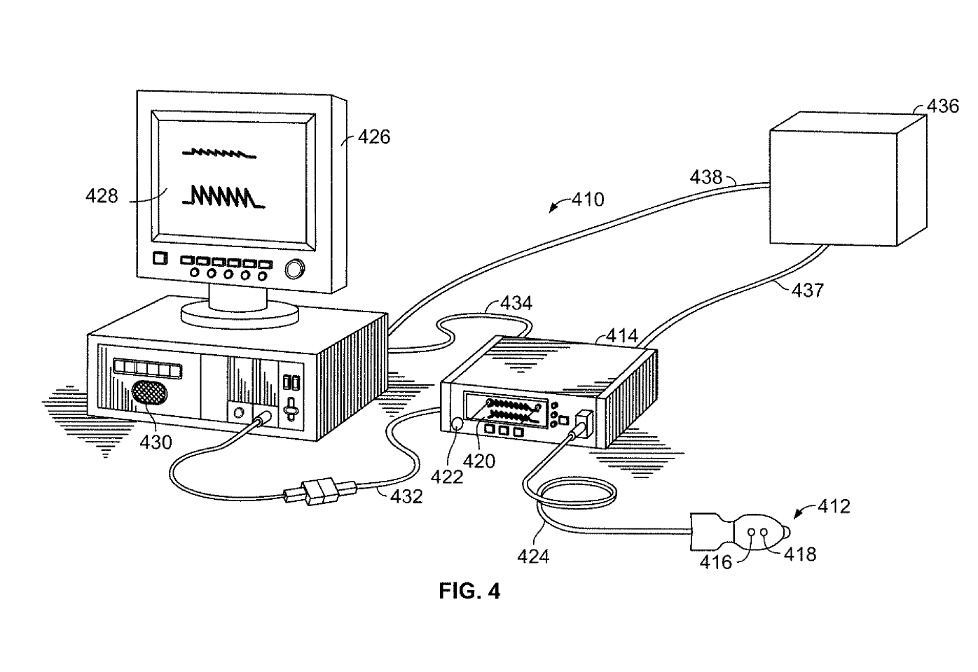

FIG. 4 illustrates an isometric view of a physiological detection system 410 according to an embodiment. For example, in the illustrated embodiment, the physiological detection system is configured as a PPG system 410. The physiological detections system 410 may be in conjunction with the physiological detector module 140 shown in FIG. 1. While the physiological system is shown and described as a PPG system 410, the system may be various other types of physiological detection systems, such as an arterial pressure detecting system including, for example, an arterial line catheter. The PPG system 410 may be a pulse oximetry system, for example. The PPG system 410 may include a PPG sensor 412 and a PPG monitor 414. The PPG sensor 412 may include an emitter 416 configured to emit light into tissue of a patient. For example, the emitter 416 may be configured to emit light at two or more wavelengths into the tissue of the patient. The PPG sensor 412 may also include a detector 418 that is configured to detect the emitted light from the emitter 416 that emanates from the tissue after passing through the tissue.

The PPG system 410 may include a plurality of sensors forming a sensor array in place of the PPG sensor 412. Each of the sensors of the sensor array may be a complementary metal oxide semiconductor (CMOS) sensor, for example. Alternatively, each sensor of the array may be a charged coupled device (CCD) sensor. In another embodiment, the sensor array may include a combination of CMOS and CCD sensors. The CCD sensor may include a photoactive region and a transmission region configured to receive and transmit, while the CMOS sensor may include an integrated circuit having an array of pixel sensors. Each pixel may include a photodetector and an active amplifier.

The emitter 416 and the detector 418 may be configured to be located at opposite sides of a digit, such as a finger or toe, in which case the light that is emanating from the tissue passes completely through the digit. The emitter 416 and the detector 418 may be arranged so that light from the emitter 416 penetrates the tissue and is reflected by the tissue into the detector 418, such as a sensor designed to obtain pulse oximetry data.

The sensor 412 or sensor array may be operatively connected to and draw power from the monitor 414. Optionally, the sensor 412 may be wirelessly connected to the monitor 414 and include a battery or similar power supply (not shown). The monitor 414 may be configured to calculate physiological parameters based at least in part on data received from the sensor 412 relating to light emission and detection. Alternatively, the calculations may be performed by and within the sensor 412 and the result of the oximetry reading may be passed to the monitor 414. Additionally, the monitor 414 may include a display 420 configured to display the physiological parameters or other information about the PPG system 410. The monitor 414 may also include a speaker 422 configured to provide an audible sound that may be used in various other embodiments, such as for example, sounding an audible alarm in the event that physiological parameters are outside a predefined normal range.

The sensor 412, or the sensor array, may be communicatively coupled to the monitor 414 via a cable 424. Alternatively, a wireless transmission device (not shown) or the like may be used instead of, or in addition to, the cable 424.

The PPG system 410 may also include a multi-parameter workstation 426 operatively connected to the monitor 414. The workstation 426 may be or include a computing sub-system 430, such as standard computer hardware. The computing sub-system 430 may include one or more modules and control units, such as processing devices that may include one or more microprocessors, microcontrollers, integrated circuits, memory, such as read-only and/or random access memory, and the like. The workstation 426 may include a display 428, such as a cathode ray tube display, a flat panel display, such as a liquid crystal display (LCD), light-emitting diode (LED) display, a plasma display, or any other type of monitor. The computing sub-system 430 of the workstation 426 may be configured to calculate physiological parameters and to show information from the monitor 414 and from other medical monitoring devices or systems (not shown) on the display 428. For example, the workstation 426 may be configured to display an estimate of a patient's blood oxygen saturation generated by the monitor 414 (referred to as an SpO₂ measurement), pulse rate information from the monitor 414 and blood pressure from a blood pressure monitor (not shown) on the display 428.

The monitor 414 may be communicatively coupled to the workstation 426 via a cable 432 and/or 434 that is coupled to a sensor input port or a digital communications port, respectively and/or may communicate wirelessly with the workstation 426. Additionally, the monitor 414 and/or workstation 426 may be coupled to a network to enable the sharing of information with servers or other workstations. The monitor 414 may be powered by a battery or by a conventional power source such as a wall outlet.

The PPG system 410 may also include a fluid delivery device 436 that is configured to deliver fluid to a patient. The fluid delivery device 436 may be an intravenous line, an infusion pump, any other suitable fluid delivery device, or any combination thereof that is configured to deliver fluid to a patient. The fluid delivered to a patient may be saline, plasma, blood, water, any other fluid suitable for delivery to a patient, or any combination thereof. The fluid delivery device 436 may be configured to adjust the quantity or concentration of fluid delivered to a patient.

The fluid delivery device 436 may be communicatively coupled to the monitor 414 via a cable 437 that is coupled to a digital communications port or may communicate wirelessly with the workstation 426. Alternatively, or additionally, the fluid delivery device 436 may be communicatively coupled to the workstation 426 via a cable 438 that is coupled to a digital communications port or may communicate wirelessly with the workstation 426. Alternatively or additionally, the fluid delivery device 436 may be communicatively coupled to one or more other aspects of a fluid responsiveness determination system, such as a fluid responsiveness analysis module or ventilator unit similar to those discussed elsewhere herein.

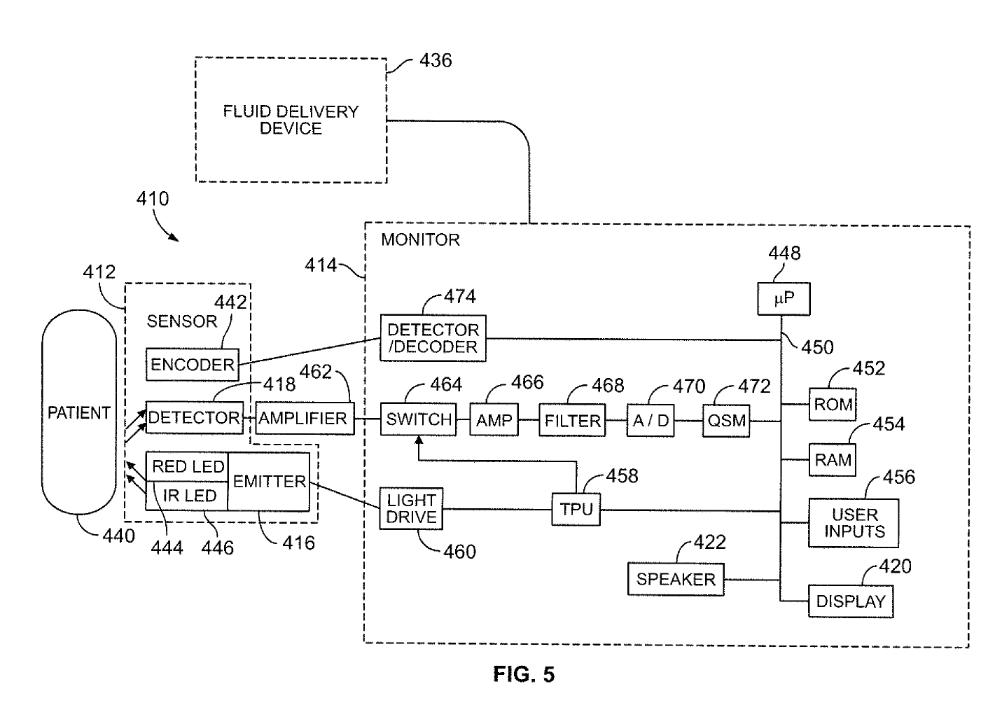

FIG. 5 illustrates a simplified block diagram of the PPG system 410, according to an embodiment. When the PPG system 410 is a pulse oximetry system, the emitter 416 may be configured to emit at least two wavelengths of light (for example, red and infrared) into tissue 440 of a patient. Accordingly, the emitter 416 may include a red light-emitting light source such as a red light-emitting diode (LED) 444 and an infrared light-emitting light source such as an infrared LED 446 for emitting light into the tissue 440 at the wavelengths used to calculate the patient's physiological parameters. For example, the red wavelength may be between about 600 nm and about 700 nm, and the infrared wavelength may be between about 800 nm and about 1000 nm. In embodiments where a sensor array is used in place of single sensor, each sensor may be configured to emit a single wavelength. For example, a first sensor may emit a red light while a second sensor may emit an infrared light.

As discussed above, the PPG system 410 is described in terms of a pulse oximetry system. However, the PPG system 410 may be various other types of systems. For example, the PPG system 410 may be configured to emit more or less than two wavelengths of light into the tissue 440 of the patient. Further, the PPG system 410 may be configured to emit wavelengths of light other than red and infrared into the tissue 440. As used herein, the term “light” may refer to energy produced by radiative sources and may include one or more of ultrasound, radio, microwave, millimeter wave, infrared, visible, ultraviolet, gamma ray or X-ray electromagnetic radiation. The light may also include any wavelength within the radio, microwave, infrared, visible, ultraviolet, or X-ray spectra, and that any suitable wavelength of electromagnetic radiation may be used with the system 410. The detector 418 may be configured to be specifically sensitive to the chosen targeted energy spectrum of the emitter 416.

The detector 418 may be configured to detect the intensity of light at the red and infrared wavelengths. Alternatively, each sensor in the array may be configured to detect an intensity of a single wavelength. In operation, light may enter the detector 418 after passing through the tissue 440. The detector 418 may convert the intensity of the received light into an electrical signal. The light intensity may be directly related to the absorbance and/or reflectance of light in the tissue 440. For example, when more light at a certain wavelength is absorbed or reflected, less light of that wavelength is received from the tissue by the detector 418. After converting the received light to an electrical signal, the detector 418 may send the signal to the monitor 414, which calculates physiological parameters based on the absorption of the red and infrared wavelengths in the tissue 440.

In an embodiment, an encoder 442 may store information about the sensor 412, such as sensor type (for example, whether the sensor is intended for placement on a forehead or digit) and the wavelengths of light emitted by the emitter 416. The stored information may be used by the monitor 414 to select appropriate algorithms, lookup tables and/or calibration coefficients stored in the monitor 414 for calculating physiological parameters of a patient. The encoder 442 may store or otherwise contain information specific to a patient, such as, for example, the patient's age, weight, and diagnosis. The information may allow the monitor 414 to determine, for example, patient-specific threshold ranges related to the patient's physiological parameter measurements, and to enable or disable additional physiological parameter algorithms. The encoder 442 may, for instance, be a coded resistor that stores values corresponding to the type of sensor 412 or the types of each sensor in the sensor array, the wavelengths of light emitted by emitter 416 on each sensor of the sensor array, and/or the patient's characteristics. Optionally, the encoder 442 may include a memory in which one or more of the following may be stored for communication to the monitor 414: the type of the sensor 412, the wavelengths of light emitted by emitter 416, the particular wavelength each sensor in the sensor array is monitoring, a signal threshold for each sensor in the sensor array, any other suitable information, or any combination thereof.

Signals from the detector 418 and the encoder 442 may be transmitted to the monitor 414. The monitor 414 may include a general-purpose control unit, such as a microprocessor 448 connected to an internal bus 450. The microprocessor 448 may be configured to execute software, which may include an operating system and one or more applications, as part of performing the functions described herein. A read-only memory (ROM) 452, a random access memory (RAM) 454, user inputs 456, the display 420, and the speaker 422 may also be operatively connected to the bus 450.

The RAM 454 and the ROM 452 are illustrated by way of example, and not limitation. Any suitable computer-readable media may be used in the system for data storage. Computer-readable media are configured to store information that may be interpreted by the microprocessor 448. The information may be data or may take the form of computer-executable instructions, such as software applications, that cause the microprocessor to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

The monitor 414 may also include a time processing unit (TPU) 458 configured to provide timing control signals to a light drive circuitry 460, which may control when the emitter 416 is illuminated and multiplexed timing for the red LED 444 and the infrared LED 446. The TPU 458 may also control the gating-in of signals from the detector 418 through an amplifier 462 and a switching circuit 464. The signals are sampled at the proper time, depending upon which light source is illuminated. The received signal from the detector 418 may be passed through an amplifier 466, a low pass filter 468, and an analog-to-digital converter 470. The digital data may then be stored in a queued serial module (QSM) 472 (or buffer) for later downloading to RAM 454 as QSM 472 fills up. In an embodiment, there may be multiple separate parallel paths having amplifier 466, filter 468, and A/D converter 470 for multiple light wavelengths or spectra received.

The microprocessor 448 may be configured to determine the patient's physiological parameters, such as SpO₂ and pulse rate, using various algorithms and/or look-up tables based on the value(s) of the received signals and/or data corresponding to the light received by the detector 418. The signals corresponding to information about a patient, and regarding the intensity of light emanating from the tissue 440 over time, may be transmitted from the encoder 442 to a decoder 474. The transmitted signals may include, for example, encoded information relating to patient characteristics. The decoder 474 may translate the signals to enable the microprocessor 448 to determine the thresholds based on algorithms or look-up tables stored in the ROM 452. The user inputs 456 may be used to enter information about the patient, such as age, weight, height, diagnosis, medications, treatments, and so forth. The display 420 may show a list of values that may generally apply to the patient, such as, for example, age ranges or medication families, which the user may select using the user inputs 456.

The fluid delivery device 436 may be communicatively coupled to the monitor 414. The microprocessor 448 may determine the patient's physiological parameters, such as a change or level of fluid responsiveness, and display the parameters on the display 420. In an embodiment, the parameters determined by the microprocessor 448 or otherwise by the monitor 414 may be used to adjust the fluid delivered to the patient via fluid delivery device 436.

As noted, the PPG system 410 may be a pulse oximetry system. A pulse oximeter is a medical device that may determine oxygen saturation of blood. The pulse oximeter may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of a patient. Pulse oximeters typically measure and display various blood flow characteristics including, but not limited to, the oxygen saturation of hemoglobin in arterial blood.

A pulse oximeter may include a light sensor, similar to the sensor 412, that is placed at a site on a patient, typically a fingertip, toe, forehead or earlobe, or in the case of a neonate, across a foot. The pulse oximeter may pass light using a light source through blood perfused tissue and photoelectrically sense the absorption of light in the tissue. For example, the pulse oximeter may measure the intensity of light that is received at the light sensor as a function of time. A signal representing light intensity versus time or a mathematical manipulation of this signal (for example, a scaled version thereof, a log taken thereof, a scaled version of a log taken thereof, and/or the like) may be referred to as a PPG signal. In addition, the term “PPG signal,” as used herein, may also refer to an absorption signal (for example, representing the amount of light absorbed by the tissue) or any suitable mathematical manipulation thereof. The light intensity or the amount of light absorbed may then be used to calculate the amount of the blood constituent (for example, oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with lower oxygen saturation. By comparing the intensities of two wavelengths at different points in the pulse cycle, it is possible to estimate the blood oxygen saturation of hemoglobin in arterial blood.

The PPG system 410 and pulse oximetry are further described in United States Patent Application Publication No. 2012/0053433, entitled “System and Method to Determine SpO₂ Variability and Additional Physiological Parameters to Detect Patient Status,” United States Patent Application Publication No. 2010/0324827, entitled “Fluid Responsiveness Measure,” and United States Patent Application Publication No. 2009/0326353, entitled “Processing and Detecting Baseline Changes in Signals,” all of which are hereby incorporated by reference in their entireties.

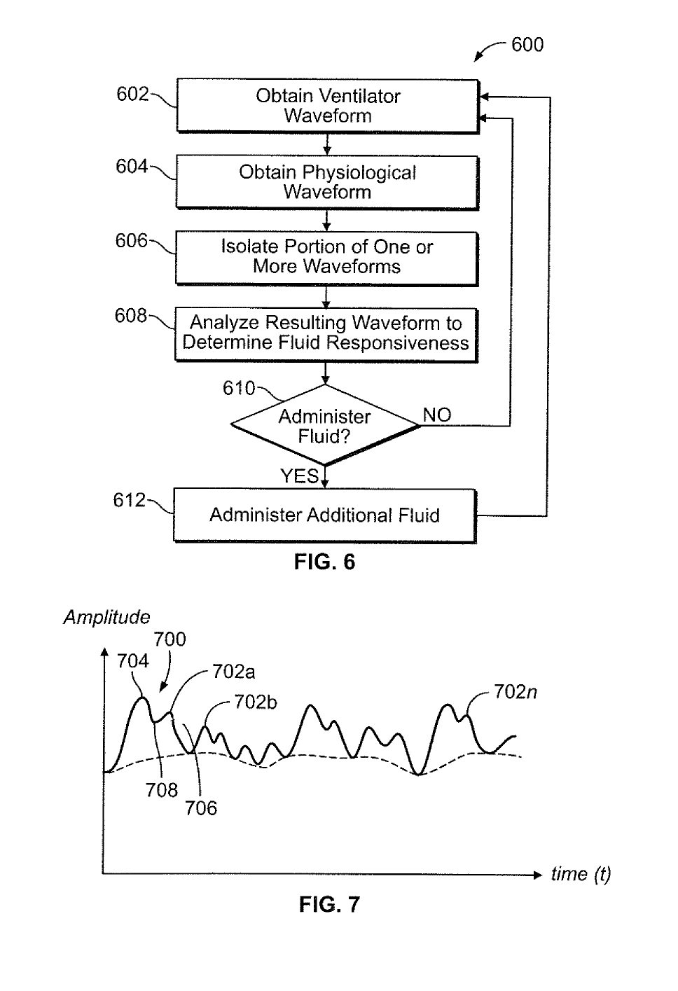

Certain embodiments provide a method for determining fluid responsiveness of a ventilated patient. For example, FIG. 6 provides a flowchart of a method 600 for determining fluid responsiveness in accordance with various embodiments. In various embodiments, certain steps may be omitted or added, certain steps may be combined, certain steps may be performed simultaneously, or concurrently, certain steps may be split into multiple steps, certain steps may be performed in a different order, or certain steps or series of steps may be re-performed in an iterative fashion. The method 600 may be performed, for example, in association with aspects, components, systems, and/or methods such as those discussed elsewhere herein.

For example, in the embodiment depicted in FIG. 6, at 602, a ventilator waveform (see, e.g., FIG. 2) is obtained. The ventilator waveform is representative of a ventilation activity or an output of a ventilator operably connected to a patient for whom fluid responsiveness is to be determined. For example, the ventilator waveform may be constructed from ventilator information representative of the ventilation activity or output collected or detected by one or more sensors. The ventilator information, for example, may describe the pressure, volume, or flow provided by a ventilator to a ventilator patient. The ventilator information may constitute all or a part of the ventilator waveform (e.g. the information may be in the form of a waveform), or the ventilator waveform may be otherwise derived from the ventilator information in either a raw or modified form (e.g. by filtering or normalization).

The ventilator information may be provided by one or more sensors or detectors positioned inside of a ventilator unit, monitor, or console, and/or positioned proximate to a breathing circuit external to a ventilator unit (e.g. a valve positioned on a line (e.g. an inspiration conduit) interposed between the ventilator unit and the patient). A sensor, detector, or transducer for providing ventilator information may be part of or otherwise associated with a ventilator unit, or may be separate. In embodiments, the ventilator waveform may be obtained directly from a ventilator unit. In other embodiments, the ventilator waveform may be obtained directly from a sensor or detector external to the ventilator unit or from a processing unit associated with the sensor or detector. In still other embodiments, the ventilator waveform may be obtained by a computation using ventilator information received from a ventilator unit, a sensor, or a sensor processing unit. For example, a processing unit configured to determine fluid responsiveness may receive ventilator information from a ventilator unit or sensor and construct the ventilator waveform using the received ventilator information.

The ventilator information and/or ventilator waveform may be obtained directly or indirectly from a sensor or detector. For example, a ventilator pressure may be directly measured by a pressure transducer associated with the ventilator. As another example, a ventilator flow may be directly measured using a flow meter associated with the ventilator. As an example of an indirectly measured type of ventilator information, a ventilator volume may be determined by integrating a directly measured flow (as flow is a change in volume over time, a volume may be obtained by integrating the flow). Further still, the ventilator information may be obtained substantially continuously, or, additionally or alternatively, at predetermined intervals. The ventilator information may describe one or more ventilator cycles, or may describe only a portion of one or more ventilator cycles. For example, the ventilator information may include a measurement or indication of positive end expiratory pressure (PEEP).

As discussed above, the ventilator waveform may correspond to a single measured or determined characteristic (e.g. ventilator pressure, ventilator volume, ventilator flow, PEEP), or may be assembled from a composite of measured or determined characteristics. Further, the ventilator waveform may be constructed by modifying, normalizing, or adjusting a waveform. For example, the ventilator waveform may be normalized, or as another example, may be synchronized to another waveform, for example by adding a time delay.

As also discussed above, the ventilator waveform may also be synchronized to another waveform, for example, by adding a time delay to a measured or determined waveform. For example, a ventilator waveform corresponding to pressure may be described as C(t)=P(t+d), where C is a function describing the ventilator waveform at a given time t, P is the pressure, and d is a time delay. Similarly, in accordance with embodiments, a ventilator waveform may be described as C(t)=V(t+d), where C is a function describing the ventilator waveform at a given time t, V is the ventilator volume, and d is a time delay. The ventilator waveform may be synchronized with another waveform, for example, by identifying a portion of the ventilator waveform corresponding to a portion of a physiological process such as a point in the respiratory cycle, identifying a portion of the additional waveform (for example a physiological or circulatory waveform discussed below) corresponding to the same portion of the physiological process, and synchronizing the two waveforms using the identified points in each waveform.

For example, with respect to ventilator waveforms corresponding to ventilator pressure or volume, features such as peaks or zeros of a waveform may be used to identify a portion of the wave with a portion of a physiological cycle. Further, derivatives or other mathematical manipulations of a waveform to identify portions of a waveform associated with a given portion of a physiological cycle. For example, the variation of pressure with time (e.g. dP/dt) or the variation of flow with time (e.g. DF/dt) may be identified to synchronize with boundaries between inspiratory and expiratory phases. As another example, with respect to ventilator waveforms corresponding to ventilator volume, features of a waveform describing the variation of volume with time (e.g. dV/dt) may be identified to synchronize with lower or upper inflection points.

The ventilator waveform may be formed or constructed from a composite of different types of measured or detected ventilator information or waveforms. Generally speaking, as also discussed above, a function C(t) describing the ventilator waveform is selected to define a signal (either composite or otherwise) that is best correlated to fluid responsiveness. Such a function C(t) may for example, be determined experimentally through clinical studies. The function C(t) may vary across different patient types or populations, and may also vary based on the type of equipment (e.g. ventilator or sensors) or combination of equipment.

At 604, a physiological waveform (see, e.g., FIG. 7) is obtained. The physiological waveform is representative of a physiological activity or process of a patient for whom fluid responsiveness is to be determined. For example, the physiological waveform may be constructed from physiologic information representative of the physiological activity or process collected or detected by one or more sensors. The physiological information, for example, may include circulatory information that describes a circulatory activity or process of the patient. The physiologic information may constitute all or a part of the physiologic waveform (e.g. the information may be in the form of a waveform) or the physiologic waveform may be otherwise derived from the physiologic information in either a raw or modified form (e.g. by filtering or normalization).

For example, the circulatory information may correspond to a level of blood within tissue. In embodiments, the circulatory information includes PPG information obtained from a sensor or detector such as a pulse oximeter positioned at a predetermined position on a patient, for example a fingertip. As another example, the circulatory information may include blood pressure information. For instance, the blood pressure information may correspond to a blood pressure waveform constructed from readings taken with an arterial line catheter. A circulatory or other physiological waveform may be constructed directly from readings taken from a sensor or detector to provide a raw waveform, or information obtained from a sensor or detector may be modified or adjusted, for example, by filtering and/or normalizing such information to construct a processed waveform. The sensor or detector may be dedicated for use exclusively in connection with determination of fluid responsiveness, or information from the sensor or detector may be shared with other systems or otherwise used for additional purposes. In embodiments, more than one sensor or detector, or more than one type of sensor or detector may be used to collect physiological information and to obtain a physiological waveform. Physiological sensors can be invasively placed (e.g. a catheter) or non-invasively placed (e.g. a pulse oximeter).

In general, a PPG waveform includes an AC physiological component related to cardiac synchronous changes in the blood volume with each heartbeat. The AC component is typically superimposed on a DC baseline that may be related to respiration, sympathetic nervous system activity, and thermoregulation. In some embodiments, a circulatory waveform is obtained by processing an obtained PPG waveform, for example, to remove high frequency artifacts and/or to remove a DC offset. For example, in some embodiments, the PPG waveform may be filtered to remove high frequency offsets. As another example, additionally or alternatively, in some embodiments the PPG waveform may be normalized by a DC value to provide a unit-less modulation depth that is robust to changes in sensor configuration. Thus, a physiological waveform may be obtained by first obtaining a raw waveform and subsequently processing the raw waveform.

FIG. 7 illustrates an example of a circulatory waveform 700, in accordance with an embodiment. Namely, FIG. 7 illustrates a PPG signal 700 over time, according to an embodiment. The PPG signal 700 is an example of a physiological signal. However, embodiments may be used in relation to various other physiological signals, such as a respiratory signal (e.g. a respiratory waveform as discussed above). Certain general principles discussed below in connection with the PPG signal 700 may also apply to other physiological signals. The PPG signal 700 may be determined, formed, and displayed as a waveform by the monitor 414 (shown in FIG. 4) that receives signal data from the PPG sensor 412 (shown in FIG. 4). For example, the monitor 414 may receive signals from the PPG sensor 412 positioned on a finger of a patient. The monitor 414 processes the received signals, and displays the resulting PPG signal 700 on the display 428 (shown in FIG. 4).

The PPG signal 700 may include a plurality of pulses 702a-702n over a predetermined time period. The time period may be a fixed time period, or the time period may be variable. Moreover, the time period may be a rolling time period, such as a 5 second rolling timeframe.

Each pulse 702a-702n may represent a single heartbeat and may include a pulse-transmitted or primary peak 704 separated from a pulse-reflected or trailing peak 706 by a dichrotic notch 708. The primary peak 704 represents a pressure wave generated from the heart to the point of detection, such as in a finger where the PPG sensor 412 (shown in FIG. 4) is positioned. The trailing peak 706 represents a pressure wave that is reflected from the location proximate where the PPG sensor 412 is positioned back toward the heart. One or more features of the PPG signal 700, such as one or more trailing peaks 706 and one or more primary peaks 704, may be used to identify a portion of a PPG signal corresponding to a physiological cycle. Similarly, a signal derived from the PPG signal 700 (e.g. a derivative or integral of the PPG signal 700) may have features, such as one or more peaks, that may be correlated to a physiological cycle. By correlating a feature (e.g. a peak) of the PPG signal 700 (or a signal derived from the PPG signal) with a corresponding feature of another signal and adjusting the PPG signal or the additional signal so that the corresponding features align, the PPG signal and the additional signal may be synchronized.

As another example, a circulatory waveform may be obtained by measuring arterial line (A-Line) pressure. For example, arterial line pressure may be measured to obtain a waveform by placing a cannula (e.g. an arterial catheter) into an artery. The cannula is operably connected to a fluid filled system which in turn is operably connected to a pressure transducer. Pressure may then be substantially continuously monitored and a waveform of arterial pressure obtained.

The circulatory waveform (e.g. PPG signal 700) may be synchronized to the ventilator waveform as discussed above, for example, by adding a time delay or otherwise aligning the phase of the ventilator and circulatory waveforms. Generally speaking, events in a first waveform (e.g. a physiological waveform) are identified and tied to events in a second waveform (e.g. a ventilator waveform), and one or both of the first and second waveforms are adjusted so that the corresponding portions of the first and second waveform align, or so that the first and second waveform are in phase with each other. The events may be identified, for example, by identifying peaks or zeros in the waveforms themselves or in derivatives of the waveforms.

For example, the end of expiration may be identified in each of the waveforms. The end of expiration may be identified in the ventilator waveform, and a time delay for the ventilator waveform or the circulatory waveform may be applied so that the portion of the ventilator waveform corresponding to the end of expiration is aligned with a peak of a PPG waveform. In alternate embodiments, a different event may be used, or more than one type of event may be used to align two waveforms or place two waveforms in phase with each other.

In some embodiments, obtaining the ventilator waveform 602 and obtaining the physiological waveform 604 may be performed without varying the ventilator from a predetermined desired treatment operation mode. For example, a predetermined desired treatment operation mode, including settings for one or more of pressure, flow, or volume, may be selected based on desired ventilation for the patient, without regard to the determination of fluid responsiveness. The ventilator and physiological waveforms may then be obtained without deviating from the predetermined desired treatment operation mode.

Thus, in accordance with some embodiments, a patient's ventilation may be unaltered during fluid responsiveness determination. In contrast, certain known systems require that a patient's ventilation be manipulated or controlled in a way that deviates from a desired treatment setting, for example, by a series of mechanically controlled breaths, for example, 3. These known systems thus suffer from a drawback of requiring deviation from a desired treatment setting, as well as provide generally limited amounts of time from which to determine fluid responsiveness. Thus, certain embodiments of the present disclosure are configured to allow a patient's ventilation to remain at a predetermined treatment setting without any deviation required for determining fluid responsiveness based on ventilation, thereby avoiding deviation from a predetermined treatment setting as well as allowing for longer sample times, for example about a minute, during which information may be gathered to be used for determining fluid responsiveness.

In some embodiments, the ventilation may be varied from a predetermined treatment setting during data acquisition for determining fluid responsiveness. For example, the PEEP may be modulated during the obtaining of the ventilator waveform, with the ventilator waveform based entirely or partially on a variation of the PEEP over time.

In some embodiments, one or both of the ventilator or physiological information may be obtained substantially continuously, for example, in the form of time based measurements at very small intervals, or, as another example, in the form of a wave provided by a sensor or a processing unit associated with the sensor. In other embodiments, one or both of the ventilator or physiological information may be obtained at discrete intervals, for example at a predetermined portion or portions corresponding to a physiological cycle, such as a respiratory cycle. For example, information may be obtained at the end of expiration. A waveform may then be constructed describing a variance over time of a measured or determined parameter at the predetermined portion or portions corresponding to the respiratory cycle.

At 606, a portion of the obtained ventilator and physiological information and/or a waveform derived from the obtained information is isolated to separate a variability due to ventilation from other variabilities in the physiological waveform. Embodiments provide for removal of all or a portion of non-ventilator induced variabilities for improved sensitivity and accuracy of fluid responsiveness variability determinations.

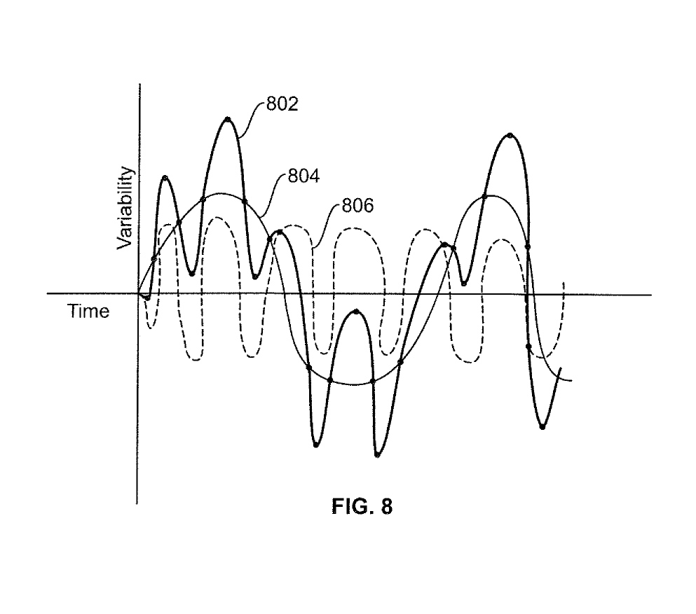

The variability in a waveform 802 may be described by FIG. 8, which illustrates variability in a waveform 802 in accordance with an embodiment. The embodiment shown in FIG. 8 is meant to be illustrative in nature and is not intended to represent any particular signal. The signal 802 represents a sensed signal that modulates from a mean value set at 0 in FIG. 8 over time. The signal 802 may be broken into components 804 and 806, each of which represent a portion of the total signal 802. In the illustrated embodiment, the signal 804 represents a portion of the signal 802 attributable to respiration-related pressure changes, while the signal 806, represented with a dashed line, represents a portion of the signal 802 attributable to all other causes. In some portions, the signals 806 and 804 are additive, and in other portions, the signals 806 and 804 cancel each other out. Due to the confounding effects of the signal portion 806, the variability in the sensed signal 802 differs in many respects to the signal 804. By isolating the change in a waveform due to the change in pressure caused by breathing (either ventilated or spontaneous), a fluid responsiveness attributable to that single cause (e.g. respiration) may be better identified to help provide an improved parameter describing fluid responsiveness.

In some embodiments, a “lock-in” technique may be employed to isolate a variation of a waveform that is synchronous with a ventilator cycle. For example, a ventilator waveform (for example, a waveform describing the operation of a variable of a ventilator such as pressure, or as another example, a composite waveform as discussed above) may be multiplied by a physiological waveform (for example a PPG waveform, which may be either raw or processed, obtained by a sensor positioned proximate to a patient's finger) to provide a mixed waveform. As also discussed above, the ventilator waveform and the physiological waveform may be synchronized before the two waveforms are multiplied. For example, a time delay may be applied to the ventilator waveform or the physiological waveform to align the waveforms based on corresponding portions of a physiological cycle, such as a breathing cycle.

In some embodiments a low pass filter (see, e.g., discussion regarding FIG. 2, above; see also discussion regarding 916 below) may then be applied to the mixed waveform. The low pass filter, for example, is selected to have a cut-off frequency lower than the ventilation rate of the ventilator. Thus, a ventilator responsiveness waveform may be obtained by multiplying the ventilator waveform by the physiological waveform to obtain a mixed waveform, and subsequently applying a low pass filter to the mixed waveform. The ventilator responsiveness waveform corresponds to an isolated variability due to the ventilator cycle, with all or a portion of other contributions to variability filtered and discarded. Next, in some embodiments, the ventilator responsiveness waveform may be normalized. For example, the ventilator responsiveness waveform may be normalized by the amplitude of the ventilator waveform. In embodiments, isolating variability due solely or predominately to ventilation allows for improved accuracy, reliability, and predictiveness of fluid responsiveness and/or fluid responsiveness determinations at lower tidal volumes and/or without manipulation of ventilator output from a desired treatment mode of operation.

At 608, the resulting ventilator responsiveness waveform is analyzed to determine fluid responsiveness. The ventilator responsiveness waveform analyzed may be, for example, the waveform resulting from the above described application of the low pass filter, or as another example, the waveform resulting from the above described normalization after application of the low pass filter. The ventilator responsiveness waveform may be analyzed, for example, to identify a unitless variability index (expressed as, for example, a fraction, a decimal number, or percentage) describing the ventilator responsiveness. For example, the ventilator responsiveness waveform variability index may be described by (VRₘₐₓ−VRₘᵢₙ)/VRₘₑₐₙ, where VR is the amplitude of the ventilator responsiveness, VRₘₐₓ is the maximum amplitude of the ventilator responsiveness waveform during a predetermined interval, VRₘᵢₙ is the minimum amplitude of the ventilator responsiveness waveform, and VRₘₑₐₙ is the mean amplitude of the ventilator responsiveness waveform. In other embodiments, other measures, indications, or expressions of variability in the ventilator responsiveness waveform may be utilized.

The resulting variability index of the ventilator responsiveness waveform, in some embodiments, may be used directly to determine whether additional fluid administration is appropriate for a given patient. For example, based on clinical studies, a threshold (or thresholds) may be established, with fluid administration appropriate (or a given quantity of fluid administration appropriate) if the threshold is met or exceeded. In some other embodiments, the resulting variability index of the ventilator responsiveness waveform may be used to identify a corresponding value of a previously recognized fluid responsiveness index, such as stroke volume variability (SVV). For example, in a clinical study, the SVV may be concurrently determined using conventional techniques and the variability index of the ventilator responsiveness waveform may be determined using, for example, techniques discussed herein, across a population of patients. By a calibration process (e.g. comparing the SVV and the variability index concurrently determined during a clinical study), a correlation between the SVV and the variability index of the ventilator responsiveness waveform may be identified. The correlation may be described, for example, by a mathematical function, or as another example, may be described in a look-up table correlating two variability indices. In still other embodiments, a description of the ventilator responsiveness waveform may be calibrated or correlated to an established variability index directly, with, for example, a function or transform determined through clinical studies correlating the ventilator responsiveness waveform and one or more established indices, such as SVV.

In some embodiments, the resulting variability index of the ventilator responsiveness waveform may be adjusted by correction factors for various demographics of patients and/or types of equipment, such as ventilators. The various computations or determinations discussed herein may be performed, for example, by a fluid responsiveness monitoring unit having a processing capability. The fluid responsiveness monitoring unit may, responsive to the determination of a fluid responsiveness index, provide a displayed indication to a practitioner. The displayed indication may include an identification of a determined fluid responsiveness index and/or a recommendation of a fluid administration activity. For example, using the determined fluid responsiveness index (and, in some embodiments, using patient information, for example, identifying a demographic group to which a patient belongs), the fluid responsiveness monitoring unit may develop a recommendation (e.g. “fluid administration not required” or “additional fluid administration indicated”) and/or may display one or more fluid responsiveness variability indices to provide information to a practitioner who will decide if additional fluid administration is performed. The fluid responsiveness monitor in some embodiments is configured as a stand-alone device that may be operably connected, for example, to the ventilator and various sensing or detecting devices, or, in some other embodiments, the fluid responsiveness monitor is incorporated into or otherwise associated with a ventilator unit and/or a ventilator display unit.

At 610, it is determined whether or not fluid is to be administered, using the determined fluid responsiveness. For example, as discussed above, a decision on whether or not to administer additional fluid may be based at least in part on whether or not a threshold of a determined fluid responsiveness index is met or exceeded.

For example, if the threshold is exceeded and it is determined to administer additional fluid, the method proceeds to 612 where additional fluid is administered. The method may then return to 602 to begin a subsequent determination if, at some point after the administration of additional fluid, still further additional fluid administration may be appropriate. If the threshold is not exceeded and it is determined not to administer additional fluid, then the method, for example, may return to 602 for ongoing monitoring to determine if fluid administration becomes appropriate at a later time.

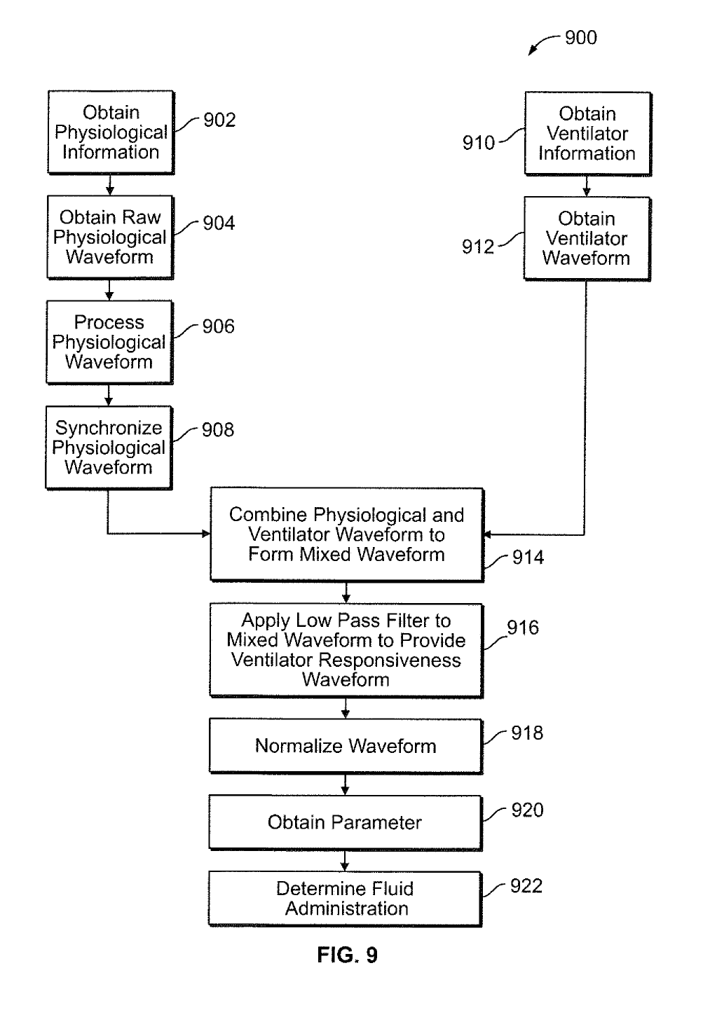

FIG. 9 illustrates a flowchart of a method 900 for determining fluid responsiveness in accordance with various embodiments. In various embodiments, certain steps may be omitted or added, certain steps may be combined, certain steps may be performed simultaneously, or concurrently, certain steps may be split into multiple steps, certain steps may be performed in a different order, or certain steps or series of steps may be re-performed in an iterative fashion. The method 900 may be performed, for example, in association with aspects, components, systems, and/or methods such as those discussed elsewhere herein.

At 902, physiological information is obtained. For example, the physiological information may include circulatory information describing a circulatory function of a patient. For example, the circulatory information may include information regarding a PPG or a blood pressure, for example a blood pressure measured by a transducer associated with an arterial line catheter. The physiological information may be collected at discrete intervals, or may be collected substantially continuously. In some embodiments, the physiological information includes PPG information, for example obtained with a pulse oximeter located proximate to a patient's finger.

At 904, a raw physiological waveform is obtained. In some embodiments, the raw physiological waveform is a PPG waveform (see, e.g., FIG. 7) that may be described as W(t). In other embodiments, for example, the raw physiological waveform may describe an arterial pressure. In various embodiments, the raw physiological waveform may be obtained in various ways. For example, the raw physiological waveform may be obtained directly from a sensor. As another example, the raw physiological waveform may be obtained, by a processing unit configured to determine fluid responsiveness, from a separate processing unit associated with a sensor obtaining the physiological information. As still another example, the raw physiological waveform may be constructed at a processing unit configured to determine fluid responsiveness (or a separate processing unit) using information (such as information recorded at discrete intervals) from a sensor or a processing unit associated with a sensor.

At 906, the physiological waveform is processed. The physiological waveform may be processed, for example, to remove noise or other artifacts, to normalize the physiological waveform, and/or to remove or isolate portions of the physiological waveform for later use. In some embodiments, a PPG waveform is processed by passing the PPG waveform through a bandpass filter and normalizing to remove a DC offset present in the raw PPG waveform due to, for example, respiration, sympathetic nervous system activity, and thermoregulation. The bandpass filter, for example, may define a band from about 0.05 Hz to about 5 Hz. In some embodiments, the raw physiological waveform may be processed at a detection processor associated with the sensor or detector that obtains the raw physiological data. Additionally or alternatively, the raw physiological waveform may be processed at a processing unit, for example, a monitor, configured to determine fluid responsiveness using, among other things, the physiological waveform.

At 908, the physiological waveform is synchronized. For example, the physiological waveform may be synchronized to a ventilator waveform. Generally speaking, the waveforms may be synchronized by identifying portions of each waveform corresponding to a given portion of a physiological cycle, such as a respiratory cycle, and aligning the identified portions of the waveforms. For example, a time delay may be applied to one waveform to synchronize with another. In some embodiments, the time delay may be a generally constant delay added to a function describing a waveform, while in other embodiments, the time delay may vary from cycle to cycle. In the depicted embodiment, the physiological waveform is synchronized to a ventilator waveform by adding a time delay, so that the physiological waveform may be considered as W(t+d). In alternate embodiments, a time delay may instead by added to a ventilator waveform to synchronize the ventilator waveform to the physiological waveform. In alternate embodiments, other techniques of synchronizing or aligning the phase of the ventilator and physiological waveforms may be employed.

At 910, ventilator information is obtained. In embodiments, the ventilator information is obtained substantially concurrently with the physiological information. Alternatively or additionally, the ventilator and physiological information may be collected and identified with a time stamp or other indicator for use in associating the two waveforms subsequently. The ventilator information may be obtained substantially continuously, for example by a substantially continuous monitoring of pressure and/or flow of a ventilator. Alternatively or additionally, the ventilator information may be obtained at discrete intervals, for example, a PEEP may be obtained at the end of expiration for a predetermined number of respiratory cycles. The ventilator information in some embodiments is obtained with a sensor or detector that is part of a ventilator unit or monitor. In some embodiments, the ventilator information may obtained by a sensor independent of the ventilator unit, for example a valve positioned proximate a breathing circuit operably connecting the ventilator unit to a ventilated patient. The ventilator information may include directly measured information, such as a ventilator flow, or may include information derived from direct measurements, such as a ventilator volume derived from a directly measured ventilator flow.

At 912, a ventilator waveform (see, e.g., FIG. 2 and related discussion) is obtained. In some embodiments, the ventilator waveform may be obtained by a fluid responsiveness processing unit that receives a waveform corresponding to ventilator output that has been obtained by a sensor or detector. In some embodiments, the ventilator waveform is obtained by constructing a waveform (e.g. the ventilator waveform is constructed by the fluid responsiveness processing unit) using data points received from a sensor or detector. The data points may be collected by the sensor or detector substantially continuously or at discrete time intervals a predetermined time apart or, as another example, corresponding to a portion or portions of a respiratory cycle. The ventilator waveform may correspond to a single measured or determined ventilator performance or operational characteristic, such as pressure, volume, flow, or PEEP.

In some embodiments, the ventilator waveform may be constructed as a composite of more than one measured or determined ventilator output, performance, or operational characteristic. (See, e.g., FIG. 2 and related discussion.) For example, the ventilator waveform may correspond to work done by the ventilator, and be constructed by multiplying the ventilator pressure by the ventilator volume. The ventilator waveform may also be formed as a composite using one or more waveforms representing a measured or determined ventilator characteristic (e.g. pressure or flow rate) and one more derivative or integral of a measured or determined ventilator characteristic (e.g. volume obtained as an integral of flow).

One or more measured or determined ventilator output waveforms (e.g. pressure, volume, flow) may be processed before a composite ventilator waveform using the one or more measured or determined ventilator output waveforms is constructed. For example, a pressure waveform to be used as part of a composite waveform may be filtered to remove noise or other artifacts, and/or to be normalized or otherwise scaled before combination with another waveform, for example, volume, to form a composite ventilator waveform.

In some embodiments, the particular form of the composite waveform may be mathematically or theoretically derived. For example, a composite ventilator waveform corresponding to work done by a ventilator may be represented as C(t)=P(t)*V(t), where C is the ventilator waveform at a given time t, P is the ventilator pressure, and V is the ventilator volume. In some embodiments, the composite waveform may be determined empirically, for example, using data collected in clinical studies. For example, the particular composite waveform components as well as any weightings or coefficients of the constituent waveforms may be determined by measuring fluid responsiveness (or another parameter) of a patient population with conventional means, and determining a composite waveform that effectively correlates or corresponds to fluid responsiveness and/or variations in fluid responsiveness. Further, the particular form of the composite waveform and/or the magnitude of coefficients or weightings for constituent waveforms of the composite waveform may vary, for example, for different ventilator modes, pressure settings, volume settings, or the like. Further still, the particular form of the composite waveform and/or the magnitude of coefficients or weightings for constituent waveforms of the composite waveform may vary by type or location of physiological sensor used, and/or by one or more patient characteristics, such as age or weight. Thus, forming the composite waveform may include selecting an appropriate composite waveform formulation, from a plurality of predetermined composite waveform formulations, based on one or more of ventilator mode, ventilator setting, patient characteristics, or sensor characteristics, and then constructing the composite waveform using the selected waveform formulation.

In some embodiments, the ventilator waveform may be obtained, by a fluid responsiveness monitor or processing unit configured to determine fluid responsiveness, by receiving the ventilator waveform from a ventilator unit or module that constructs the ventilator waveform. In some embodiments, the ventilator waveform may be obtained, by a fluid responsiveness monitor or processing unit configured to determine fluid responsiveness, by receiving the ventilator waveform from a sensing or detection unit or module that constructs the ventilator waveform using information collected by the sensing or detection unit or module. In other embodiments, the fluid responsiveness monitor or processing unit may obtain the ventilator waveform by constructing the ventilator waveform using information provided by a sensor and/or a ventilator unit. For example, a ventilator unit may provide ventilator information including PEEP recorded for each ventilator cycle to a fluid responsiveness monitor. The fluid responsiveness monitor may then construct a ventilator waveform using a variation or evolution of the PEEP over time.

At 914, the physiological waveform (e.g. (W(t+d)) and the ventilator waveform (e.g. C(t), where C is a composite waveform using one or more of pressure, volume, flow, or PEEP) are combined to form a mixed waveform. (See, e.g., FIG. 3a and related discussion.) In some embodiments, the physiological waveform and the ventilator waveform are multiplied to form the mixed waveform. For example, the mixed waveform “M” may be described as M=C(t)*W(t+d), where d is a time delay applied to the physiological waveform to synchronize the physiological waveform to the ventilator composite waveform. In alternate embodiments, the time delay may be applied to the ventilator composite waveform, while in still other embodiments, a different synchronization or phase alignment technique may be employed. Different weightings or coefficients may also be employed in other embodiments. In the depicted embodiment, the multiplication of the ventilator waveform and the physiological waveform may be performed to help identify and isolate variations in the physiological waveform induced by mechanical ventilation from variations caused by other sources.

For example, in the illustrated embodiment, at 916, a low pass filter is applied to remove portions of the mixed waveform that do not correspond to variations induced by mechanical ventilation. (See, e.g., FIG. 3b.) By multiplying the physiological waveform and the ventilator waveform to form a mixed waveform, and then applying a low pass filter to the mixed waveform to form a ventilator responsiveness waveform, portions of the mixed waveform that do not correspond to ventilator induced behavior may be removed, and portions of the mixed waveform attributable to ventilator variations may be entirely or partially isolated in the ventilator responsiveness waveform. Thus, in embodiments, such a ventilator responsiveness waveform may provide a more specific representation of the variation due to the ventilator alone, which in turn may provide improved accuracy and reliability of fluid responsiveness determinations.

At 918, the ventilator responsiveness waveform is normalized. In some embodiments, the ventilator responsiveness waveform is normalized by the amplitude of the composite ventilator waveform. For example, normalizing the ventilator responsiveness waveform by the amplitude of the composite ventilator waveform may quantify the effect of the ventilator (e.g. flow, pressure, volume) on the waveform variation obtained by the multiplication and filtering (which may be referred to as lock-in detection) discussed above.

At 920, a ventilator responsiveness parameter is obtained using the ventilator responsiveness waveform. For example, the fluid responsiveness parameter may be a unitless parameter (e.g. a percentage) describing the variability of the ventilator responsiveness waveform obtained at 916 and/or 918 above. For example, in some embodiments, a variability of the ventilator responsiveness waveform (referred to herein as a ventilator responsiveness waveform variability index) may be described as (VRₘₐₓ−VRₘᵢₙ)/VRₘₑₐₙ, where VRₘₐₓ corresponds to the maximum amplitude of the ventilator responsiveness waveform, VRₘᵢₙ corresponds to the minimum amplitude of the ventilator responsiveness waveform, and VRₘₑₐₙ corresponds to the mean amplitude of the ventilator responsiveness waveform. In alternate embodiments, other descriptions of the variability of the ventilator responsiveness waveform may be employed.

Further still, additionally or alternatively, in some embodiments, the ventilator responsiveness waveform may be used to obtain a conventionally known fluid responsiveness index, such as SVV. This may be done in one step, using information from the ventilator responsiveness waveform to directly compute the SVV. For example, clinical studies may be used to determine a relationship between the ventilator responsiveness waveform or components or aspects thereof with SVV. Such a relationship, for example, may be described by an experimentally derived formulaic relationship. As another example, a conventional fluid responsiveness index, such as SVV, may be obtained in a multi-step process. For instance, the ventilator responsiveness waveform may be analyzed to determine a variability of the ventilator responsiveness waveform, for example as discussed in the preceding paragraph. The ventilator responsiveness waveform variability index may then be converted to a conventionally known or familiar index, such as SVV. The conversion may be accomplished by a formula obtained during a calibration of the ventilator responsiveness waveform variability index to SVV performed during clinical studies. As another example, a lookup table correlating the ventilator responsiveness waveform variability index to SVV may be obtained by a calibration process in clinical studies and utilized to convert the ventilator responsiveness waveform variability index to SVV.

At 922, it is determined if additional fluid administration is appropriate. Such a determined may be made using, for example, the ventilator responsiveness waveform variability index. For example, a threshold or thresholds at which fluid administration is recommended based on the ventilator responsiveness waveform variability index may be determined in clinical studies. As another example, the determination may be made based on a conventional index, such as SVV, with the SVV determined using the ventilator responsiveness waveform or ventilator responsiveness waveform variability index as discussed above. For example, a fluid responsiveness monitor or processing unit that has determined one or more fluid responsiveness parameters (e.g. the ventilator responsiveness waveform variability index, SVV, PVI, or PPV) may display the determined parameter and/or a recommendation for fluid administration based on a predetermined criterion (e.g. a threshold). A practitioner may then determine whether additional fluid administration is appropriate, and administer additional fluid if appropriate.

The method 900 may be performed in an iterative or ongoing fashion. For example, a determined fluid responsiveness index may be substantially continuously displayed, and an alarm or other signal may be activated or otherwise communicated if a threshold is crossed that indicates additional fluid administration is appropriate. In some embodiments, a fluid responsiveness may be determined periodically (e.g. every minute or other predetermined time period) using information collected during the previous minute or other time period) or may be determined on a rolling basis.

Thus, embodiments of the present disclosure provide for the isolation of ventilation variability (variability caused by ventilation in a waveform) from other variability (variability caused by one or more other sources of potential variability), thereby allowing for a more controlled study and determination of fluid responsiveness. For example, embodiments provide systems and methods that are configured to more accurately determine a fluid responsiveness index or indices. Further, embodiments provide systems and methods that are configured to allow a determination of fluid responsiveness at relatively low tidal volume ventilation. Also, embodiments provide systems and methods configured to determine fluid responsiveness for smaller variations of waveforms.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer.”

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. For example, a module or system may include a computer processor, controller, or other logic-based device that performs operations based on instructions stored on a tangible and non-transitory computer readable storage medium, such as a computer memory. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A system for determining fluid responsiveness, the system comprising:
a ventilator detection module configured to be operably connected to a ventilator, the ventilator detection module configured to detect ventilator information representative of a ventilation activity performed by the ventilator on a ventilated patient, the ventilator information corresponding to one or more of a pressure of the ventilator, a flow of the ventilator, or a volume of the ventilator;
a circulatory detection module configured to detect circulatory information representative of the circulation of the ventilated patient; and
a fluid responsiveness analysis module configured to obtain a ventilator waveform based at least in part on the ventilator information, obtain a circulatory waveform based at least in part on the circulatory information, combine the ventilator waveform and the circulatory waveform to provide a mixed waveform, and isolate a portion of the mixed waveform to identify a ventilator responsiveness waveform representative of an effect of the ventilator on the mixed waveform.

2. The system of claim 1, wherein the fluid responsiveness analysis module is further configured to determine a fluid responsiveness parameter representative of fluid responsiveness of the ventilated patient using the ventilator responsiveness waveform.

3. The system of claim 1, wherein the fluid responsiveness analysis module is further configured to combine the ventilator waveform and the circulatory waveform by multiplication and to apply a low-pass filter to the mixed waveform to isolate the portion of the mixed waveform.

4. The system of claim 1, wherein the circulatory detection module comprises a pulse oximetry sensor configured to provide photoplethysmographic information representative of a photopleythsmographic waveform of the ventilated patient.

5. The system of claim 1, wherein the circulatory detection module comprises an arterial line catheter and a pressure transducer, the pressure transducer configured to be associated with the arterial line catheter and to provide blood pressure information representative of a blood pressure waveform of the ventilated patient.

6. The system of claim 1, wherein the ventilator information comprises information corresponding to a variation in positive end expiratory pressure (PEEP) of the ventilator.

7. A method for determining fluid responsiveness of a ventilated patient, the method comprising:
obtaining a ventilator waveform representative of a ventilation activity performed by a ventilator on a ventilated patient;
obtaining a circulatory waveform representative of the circulation of the ventilated patient, the circulatory waveform based on information provided by a circulatory detection module; and
combining, at a processing module, the ventilator waveform and the circulatory waveform to provide a mixed waveform;
isolating, at the processing module, a portion of the mixed waveform to provide a ventilator responsiveness waveform representative of an effect of the ventilator on the mixed waveform.

8. The method of claim 7, further comprising determining, at the processing module, a fluid responsiveness parameter representative of fluid responsiveness of the ventilated patient using the ventilator responsiveness waveform.

9. The method of claim 7, wherein combining the ventilator waveform and the circulatory waveform comprises multiplying the ventilator waveform and the circulatory waveform, and wherein isolating the portion of the mixed waveform comprises applying a low-pass filter to the mixed waveform to isolate the portion of the mixed waveform.

10. The method of claim 7 further comprising normalizing the ventilator responsiveness waveform by an amplitude of the ventilator waveform.

11. The method of claim 7, further comprising constructing the ventilator waveform as a composite waveform using at least two of a ventilator pressure waveform, a ventilator flow waveform, or a ventilator volume waveform.

12. The method of claim 7, further comprising constructing the ventilator waveform using a variation in positive end expiratory pressure (PEEP) of the ventilator.

13. The method of claim 7, wherein the obtaining the ventilator waveform and the circulatory waveform are performed without varying operation of the ventilator from a desired treatment operation mode, wherein the desired treatment operation mode is determined without respect to the determining of the fluid responsiveness parameter.

14. A tangible and non-transitory computer readable medium comprising one or more computer software modules configured to direct a processor to:
obtain a ventilator waveform representative of a ventilation activity performed by a ventilator on a ventilated patient;
obtain a circulatory waveform representative of the circulation of the ventilated patient, the circulatory waveform based on information provided by a circulatory detection module;
combine the ventilator waveform and the circulatory waveform to provide a mixed waveform; and
isolate a portion of the mixed waveform to provide a ventilator responsiveness waveform representative of an effect of the ventilator on the mixed waveform.

15. The computer readable medium of claim 14, wherein the computer readable medium is further configured to direct the processor to determine a fluid responsiveness parameter representative of fluid responsiveness of the ventilated patient using the ventilator responsiveness waveform.

16. The computer readable medium of claim 14, wherein the computer readable medium is further configured to direct the processor to combine the ventilator waveform and the circulatory waveform by multiplication and to apply a low-pass filter to the mixed waveform to isolate the portion of the mixed waveform.

17. The computer readable medium of claim 14, wherein the computer readable medium is further configured to direct the processor to normalize the ventilator responsiveness waveform by an amplitude of the ventilator waveform.

18. The computer readable medium of claim 14, wherein the computer readable medium is further configured to direct the processor to construct the ventilator waveform as a composite waveform using at least two of a ventilator pressure waveform, a ventilator flow waveform, or a ventilator volume waveform.

19. The computer readable medium of claim 14, wherein the computer readable medium is further configured to direct the processor to construct the ventilator waveform using a variation in positive end expiratory pressure (PEEP) of the ventilator.

20. The computer readable medium of claim 14, wherein the computer readable medium is further configured to direct the processor to obtain the ventilator waveform and the circulatory waveform without varying operation of the ventilator from a desired treatment operation mode, wherein the desired treatment operation mode is determined without respect to the determining of the fluid responsiveness parameter.
